(19) United States
(12) Patent Application Publication
(10) Pub. No.: US 2004/0258210 A1
(43) Pub. Date: Dec. 23, 2004

(54) X-ray apparatus and method to produce a surface image

(76) Inventor: Dieter Ritter, Erlangen (DE)

Correspondence Address: SCHIFF HARDIN LLP, Patent Department, 6600 Sears Tower, 233 South Wacker Drive, Chicago, IL, 60606, US

(21) Appl. No.: 10/824,225

(22) Filed: Apr. 14, 2004

(30) Foreign Application Priority Data
Apr. 14, 2003 (DE) 103 17 137.1

(51) Int. Cl. G21K 1/12; A61B 6/00; H05G 1/60; G01N 23/00

(52) U.S. Cl. 378/198

(57) ABSTRACT

In a method and x-ray apparatus to produce a surface image of an examination subject, wherein the x-ray apparatus that comprises a carrier support for an x-ray system including an x-ray source and a radiation detector, the carrier support is moved relative to the examination subject during the acquisition of a series of 2D projections of the examination subject. A 3D sensor is mounted on the carrier support that acquires an image dataset of the examination subject during movement of the carrier support relative to the examination subject. The image dataset represents an image of at least one part of the surface of the examination subject. The invention also concerns an x-ray apparatus (1) with which the inventive method can be implemented.

### BACKGROUND OF THE INVENTION

1. Field of the Invention

The present invention concerns an x-ray apparatus of the type having a carrier support on which an x-ray system, including an x-ray source and a radiation detector, is mounted. The invention also concerns a method to produce a surface image of an examination subject with such an x-ray apparatus.

2. Description of the Prior Art

In addition to x-ray exposures, optical shape recognition has great importance, in particular in plastic surgery. Optical 3D sensors used for this can in principle be divided into two classes: passive methods (stereo, shading, contour) and active methods (laser scanner, moiré, coherence radar, propagation). The former are, as a rule, technically simpler to realize. In contrast, methods with active illumination have greater precision and are more robust. 3D sensors are, among other things, specified in S. Blossey, G. Häusler, F. Stockinger, “A Simple and Flexible Calibration Method for Range Sensors”, Int. Conf. of the ICO, Kyoto, April 1994, page 62, R. G. Dorsch, G. Häusler, J. M. Herrmann, “Laser triangulation: fundamental uncertainty in distance measurement”, Applied Optics, Vol. 33, No. 7, March 1994, pages 1306-1314, T. Dresel, G. Häusler, H. Venzke, “Three-dimensional sensing of rough surfaces by coherence radar”, Applied Optics, Vol. 31, No. 7, March 1992, pages 919-925, K. Engelhardt, G. Häusler, “Aquisition of 3-D data by focus sensing”, Applied Optics, Vol.27, No. 22, November 1988, pages 4684-4689, M. Gruber, G. Häusler, “Simple, robust and accurate phase-measuring triangulation”, Optik, 89, No. 3, 1992, pages 118-122, G. Häusler, W. Heckel, “Light Sectioning with Large Depth and High Resolution”, Applied Optics, Vol. 27, No. 24, 15 Dec. 1988, pages 5165-5169, G. Häusler, D. Ritter, “Parallel Three-Dimensional. Sensing by Color-Coded Triangulation”, Applied Optics, Vol. 32, No. 35, 10 Dec. 1993, pages 7164-7169.

### SUMMARY OF THE INVENTION

An object of the invention provide an x-ray apparatus of the above-cited type with which a surface image of the examination subject also can be produced.

It is a further object of the invention to provide a method for generating an image of at least one part of the surface of the examination subject with an x-ray apparatus of the above-cited type.

The first object of the invention is achieved by an x-ray apparatus with a carrier support on which is an x-ray system, including an x-ray source and a radiation detector is mounted, the carrier support being movable relative to the examination subject during the acquisition of a series of 2D projections of an examination subject, and wherein a 3D sensor is mounted on the carrier support, and the carrier support can be moved relative to the examination subject for the acquisition of an image dataset with the 3D sensor, the image dataset representing an image of at least one part of the surface of the examination subject.

The inventive x-ray apparatus has a carrier support that is implemented according to an embodiment of the invention as a C-arm on which the x-ray system is mounted, i.e., the x-ray source and the radiation detector are mounted on the C-arm. If the x-ray apparatus is used to produce the series of 2D projections (from which, for example, a volume dataset of the examination subject can be calculated), then the carrier support is shifted relative to the examination subject (for example a patient) during the acquisition of the series of 2D projections. If the carrier support is a C-arm, the C-arm is shifted along its circumference (orbital motion) during the acquisition of the series of 2D projections, or the series of 2D projections is acquired during an angulation movement. According to a preferred embodiment, the inventive x-ray apparatus is an isocentric C-arm x-ray apparatus.

In addition to the x-ray system, the 3D sensor is inventively mounted on the carrier support. With the 3D sensor, an image dataset is acquired that represents at least one part of the surface of the examination subject. Similar to the acquisition of the series of 2D projections, the carrier support is shifted relative to the examination subject during the acquisition of the image dataset. The x-ray source is deactivated. It is also possible, however, to simultaneously acquire the series of 2D projections and the image dataset, thus to acquire the series of 2D projections and the image dataset, during a single shift movement of the carrier support relative to the examination subject. 3D sensors are known for example from the printed publications cited in the above. 3D sensors are necessary in order to acquire geometric data bout the surface of an examination subject in space. Optical 3D sensors are thereby characterized by their speed and their contact-free measurement principle (compare, for example, S. Blossey, G. Häusler, “Optische 3D-Sensoren und deren industrielle Anwendung”, Messtec 1/96, March 1996, pages 24-26). They serve as an object detection and localization means for acquisition of image data from all sides of the examination subject. To acquire the data, 3D data (as an alternative to the 2D grey scale value image) are processed independent of the subject reflectivity, exposure, color and perspective (and thus robustly). Depending on the task, the performance features of the sensor types that are used are determined according to the following definitions.

The data rate means the number of the subject points measured per second. Differentiation is thereby made between punctiform (for example distance sensors), linear (for example, light-section sensors) or area (for example coded light approach) 3D sensors that, depending on the evaluation method in a measurement cycle, can evaluate one measurement point, one measurement line or one measurement field up to the size of 768*512 pixels. In the latter case, currently data rates up to 5 Mhz are possible.

The longitudinal measurement uncertainty δz designates the standard deviation with which the absolute displacement of z from ∀δz can be precisely measured. It refers to different subject points of a plane to be measured. In contrast to this, the longitudinal resolution capability 1/Δx designates the relative minimum resolvable displacement change Δz of an individual subject point. Depending on the sensor principle, at present a measurement uncertainty of up to 2 μm can be realized; the resolution capability clearly be greater. For robust subject recognition tasks this value is relatively uncritical; in contrast, precise localization methods require optimally precise surface data.

The lateral resolution capability 1/Δx refers to the minimum distance Δx of two subject points that is necessary for their differentiation. Given areal 3D sensors, Δx=Δy is determined via corresponding sensor design optically calibrated in practice via the pixelation of the CCD camera chips as an acquisition sensor.

The measurement region ΔX, ΔY, ΔZ determines the size of the available measurement field and is, among other things, defined via the measurement uncertainty and the lateral resolution capability. In practice, the number of the differentiable separations presently yields ΔZ/δz=500 . . . 2000 and a scaling of the measurement volume from approximately 100³ μm³ up to approximately 500³ mm³.

For the coding of 3D information via light, various properties can be used, such as intensity, color, polarization, coherency, phase, contrast, location or transit propagation time. In practice, the most important methods can be divided according to four evaluation methods.

Active triangulation is the most frequently used method. The subject to be measured is illuminated from one direction with a light spot and observed at an angle relative to this. The height h of the subject at the illuminated location results from the location of the image on a detector. This method is, among other things, specified in R. G. Dorsch, G. Häusler, J. M. Herrmann, “Laser Triangulation: fundamental uncertainty in distance measurement”, Applied Optics, Vol. 33, No. 7, March 1994, pages 1306-1314.

Practical methods measure linearly with the aid of a laser scanner (compare G. Häusler, W. Heckel, “Light Sectioning with Large Depth and High Resolution”, Applied Optics, Vol. 27, No. 24, 15 Dec. 1988, pages 5165-5169) or areally (in parallel) by the projection of a coded light pattern (raster) on the subject. In G. Häusler, D. Ritter, “Parallel Three-Dimensional Sensing by Color-Coded Triangulation”, Applied Optics, Vol. 32, No. 35, 10 Dec. 1993, pages 7164-7169, a method is specified in which a monochromatic spectrum is projected in which the individual, adjacent scan lines are identified by color. In M. Gruber, G. Häusler, “Simple, robust and accurate phase-measuring triangulation”, Optik, No. 3, 1992, pages 118-122, a phase-measured triangulation is specified in which the phase of the projected sine grid is measured from four sequential exposures, and from this the height is determined.

In the case interference methods, a reference wave with known phase and a subject wave of unknown phase are coherently superpositioned. The height of the examination subject is reconstructed (in parallel) from the interferogram. For short-coherent light sources, the absolute surface shape can be measured via the evaluation of the correlogram. Although interference methods are precise, in practice only optically smooth surfaces can be absolutely measured. Rough subjects can also be measured with a special evaluation method as disclosed in T. Dresel, G. Häusler, H. Venzke, “Three-dimensional sensing of rough surfaces by coherence radar”, Applied Optics, Vol. 31, No. 7, March 1992, pages 919-925.

In an active focus search, the examination subject is illuminated and imaged with a light spot or other configuration. In principle, there are two types of evaluation. In the first, the subject point to be measured is mechanically back-projected; from this, the distance can be directly determined. The second method measures the contrast dependent on the distance of the object from the camera, and from this calculates the subject shape (compare K. Engelhardt, G. Häusler, “Acquisition of 3-D data by focus sensing”, Applied Optics, Vol. 27, No. 22, November 1998, pages 4684-4689.

Propagation measurement systems use the propagation speed of light. The distance can be calculated from the measurement of the duration of a reflected short light pulse. The short time measurement necessary for a high spatial resolution is possible with electronic, amplitude- or frequency-modulating methods (compare I. Moring, T. Heikkinen, R. Myllalä, “Acquisition of three-dimensional image data by a scanning laser range finder”, Opt. Eng. 28 (8), 1989, pages 897 through 902.

In a preferred embodiment, the image computer of inventive x-ray apparatus is programmed to calculate, from the series of 2D projections (that is acquired before, after or during the acquisition of the image dataset) a volume dataset of the examination subject that is fused or superimposed with the image dataset.

The aforementioned object also is achieved in accordance with the invention by a method to produce a surface image of an examination subject with an x-ray apparatus that has a carrier support for an x-ray system, including an x-ray source and a radiation detector, and the carrier support is moved relative to the examination subject during the acquisition of a series of 2D projections of the examination subject, and the carrier support is moved relative to the examination subject for the acquisition of an image dataset with a 3D sensor arranged on the carrier support, the image dataset representing at least one part of the surface of the examination subject.

### DESCRIPTION OF THE DRAWINGS

FIG. 1 shows a C-arm x-ray apparatus constructed and operating in accordance with the invention, with a patient.

FIG. 2 shows the C-arm x-ray apparatus of FIG. 1 without a patient.

### DESCRIPTION OF THE PREFERRED EMBODIMENTS

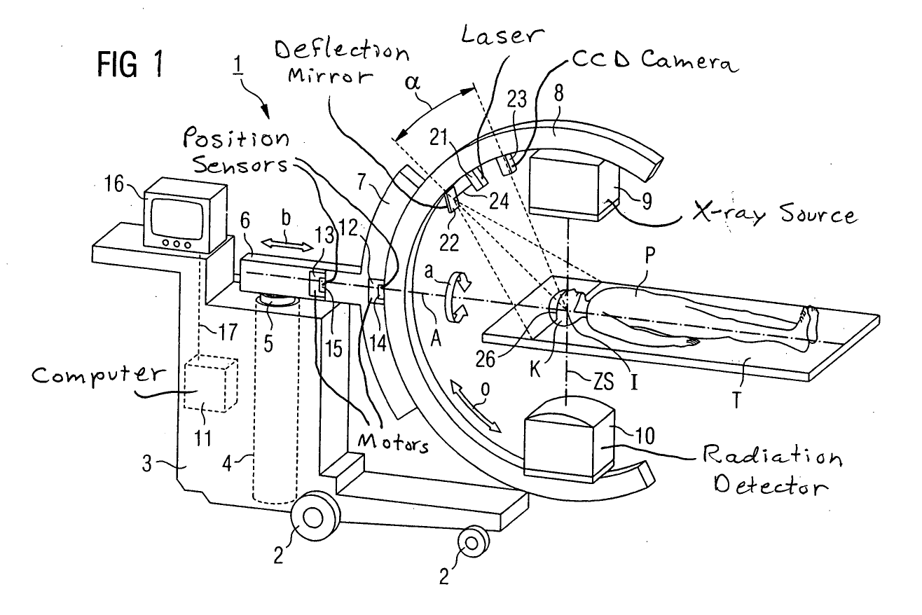

FIG. 1 schematically shows an isocentric C-arm x-ray apparatus 1. In the exemplary embodiment, the C-arm x-ray apparatus 1 has a device cart that can be moved on wheels 2. The C-arm x-ray apparatus 1 has a lifting device 4 with a column 5, schematically indicated in FIG. 1. Arranged on the column 5 is a holder 6, on which in turn is arranged a support part 7 to support a C-arm 8. The C-arm 8 carries an x-ray source 9 and a radiation detector 10 which are mounted opposite one another on the C-arm 8, such that a central beam ZS of an x-ray beam originating from the x-ray source 9 is approximately centrally incident on the detector surface of the radiation detector 10. For example, a planar image detector or an x-ray image intensifier as are generally known can be used as the radiation detector 10.

The support part 7 is held by the holder 6 so as to be rotatable in a known manner around a common axis A of the holder 6 and the support part 7 (double arrow a, angulation) and can be moved (double arrow b) in the direction of the axis A. The C-arm 8 is held in the support part 7 such that it can be displaced with regard to the isocenter I of the C-arm 8 along its circumference in the direction of the double arrow o (orbital motion).

With the lifting device 4, the C-arm 8 (that is connected with the column 5 of the lifting device 4 via the support part 7 and the holder 6) can be adjusted vertically relative to the device cart 3.

A patient P (shown schematically in FIG. 1) lies on a table T that is (likewise shown only schematically, and that is transparent for x-ray radiation) that can be adjusted vertically with a lifting device (not shown). The patient P can be examined radiologically in different manners according to the adjustment possibilities (cited previously) of the C-arm x-ray apparatus 1 of the table T, with x-ray radiation originating from the x-ray source 9 permeating the patient P with the central beam ZS and striking on the radiation detector 10.

The C-arm x-ray apparatus 1 in particular produces a volume dataset of body parts of the patient P. In the exemplary embodiment, a computer 11 is arranged in the device cart 3, the computer 11 being connected (in a known manner not shown in FIG. 1) with the radiation detector 10, and in a known manner a volume dataset of the body part to be represented is reconstructed from a series of 2D projections (acquired with the x-ray source 9 and the radiation detector 10) obtained with a displacement of the C-arm 8 around a body part of the patient P to be represented in the image. The C-arm 8 is either moved along its circumference in the direction of the double arrow o relative to the bearing part 7 or through approximately 190° with regard to the angulation axis A. Approximately 50 to 100 2D projections are acquired during the displacement. In the exemplary embodiment, the computer 11 controls the displacement of the C-arm 8 by means of an electrical drive motor 12 in the support part 7, or by means of an electrical drive motor 13 in the holder 6. The computer 11 is connected with the electrical drive motors 12 and 13 in a known manner not shown.

In order to be able to reconstruct the volume dataset from the series of 2D projections, respective position sensors (encoders) 14 and 15, which associate a position of the C-arm 8 relative to the body part to be represented with each of the 2D projections of the body part to be acquired, are integrated into the electrical drive motors 12 and 13. Projection geometries which are necessary for the reconstruction are determined from the positions identified with the sensors 14 and 15.

Due to the limited mechanical strength and resistance to deformation of the C-arm 8, the x-ray source 9 and the radiation detector 10 can easily become aligned differently relative to one another depending on the position of the C-arm 8. In the exemplary embodiment errors (resulting via the deformation of the C-arm 8) with regard to the geometry of the C-arm 8 are compensated for the most part by means of an offline calibration, for example with a calibration phantom or projection matrices. The offline calibration is implemented, for example, during the initial operation of the C-arm x-ray apparatus I or shortly before the acquisition of a series of 2D projections. An example of such an offline calibrations is specified in U.S. Pat. No. 5,923,727, cited in the preamble.

In the exemplary embodiment, a volume dataset of the head K of the patient P is prepared with the C-arm 8 (as described) moving along its circumference, and a series of 2D projections of the head K of the patient P is thereby prepared. An orbital scan thus is implemented. From the series of 20 projections, the computer 11 calculates a volume dataset from which an image is reconstructed and displayed at a monitor 16 that is connected with the computer 11 by an electrical line 17.

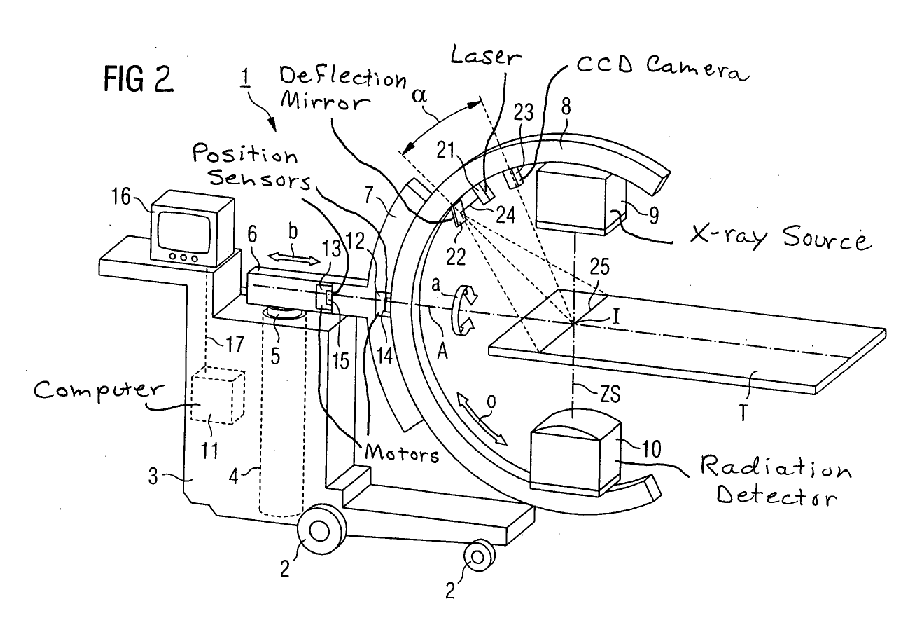

A 3D sensor also is arranged on the C-arm 8. In addition to FIG. 1, reference is also made to FIG. 2 for explaining the functioning of the 3D sensor. The C-arm x-ray apparatus 1 of FIG. 1 is likewise shown in FIG. 2, but no patient P is located on the table T.

In the exemplary embodiment, the 3D sensor is formed by a laser 21, a deflection mirror 22 and a CCD camera 23. The laser 21 is mounted on the C-arm 8 so that the laser beam originating from the laser 21 is incident on the deflection mirror 22. The deflection mirror 22 is mounted on the C-arm 8 so that it can be pivoted and, in the exemplary embodiment, is moved with an electromotor (not shown in the figures) so that what is known as a “light line” 25 (aligned parallel to the orbital rotation axis of the C-arm 8) that is emitted onto the table T (see FIG. 2) is created from the laser beam 24 for each position of the C-arm 8 relative to the device truck 3. This is acquired by the CCD camera 23 that is attached to the C-arm 8 at a triangulation angle α.

If a subject (in the exemplary embodiment, the patient P or his head K) is located on the table, a subject height line 26 (shown in FIG. 1) that is emitted on the head K of the patient P is created from the light line 25 (shown in FIG. 2). The CCD camera 21 scans the subject height line 26 at the triangulation angle α. The electrical signals from this scan are supplied to the computer 11 with which the CCD camera 21 is electrically connected in a manner not shown. From these signals, the computer 11 calculations the displacement of the subject height line 26 relative to the light line 25 associated with the current position of the C-arm 8.

In order to now obtain a 3D height image of the head surface of the patient P, thus a surface image of the head K of the patient P, the C-arm 8 is moved along its circumference with the 9 deactivated x-ray source (orbital scan). During the orbital scan, subject height lines are acquired in this manner for various positions of the C-arm 8 relative to the device carts, and the signals associated with them are forwarded to the computer 11. From the individual subject height lines the computer 11 calculates the surface image, which can be reproduced at the monitor 16.

The position of the 3D sensor must be known for the calculation of the individual surface height lines, or the surface image. Since the C-arm 8, as already noted, slightly deforms in practice, in the exemplary embodiment it undergoes an offline calibration (already specified). The position of the 3D sensor thus is sufficiently precisely known for each position of the C-arm 8, so that the surface image can be calculated.

If the patient P is aligned the same for the orbital scan to produce the volume dataset and the surface image, it is possible in a simple manner to overlap (overlay) the surface image and the x-ray image associated with the volume dataset.

It is also possible for the series of 2D projections and the scan of the patient P with the laser 21 to be implemented during exactly one orbital scan.

Although modifications and changes may be suggested by those skilled in the art, it is the intention of the inventor to embody within the patent warranted hereon all changes and modifications as reasonably and properly come within the scope of his contribution to the art.

I claim as my invention:

1. An X-ray apparatus comprising:
an x-ray imaging system comprising a carrier support with an x-ray source and a radiation detector mounted thereon at respective positions allowing an examination subject to be disposed between the x-ray source and the radiation detector;
a supporting arrangement for said carrier support for moving said carrier support relative to the examination subject for acquiring a series of 2D projections of the examination subject with the x-ray source and the radiation detector;
an optical 3D sensor mounted to said carrier support; and
said supporting arrangement for said carrier support also moving said carrier support relative to said examination subject for acquiring an image dataset with said optical 3D sensor representing at least a portion of a surface of the examination subject.

2. An X-ray apparatus as claimed in claim 1, wherein said carrier support is a C-arm.

3. An X-ray apparatus as claimed in claim 2, wherein said C-arm has a circumference, and wherein said supporting arrangement moves said C-arm along said circumference during acquisition of said series of 2D projections.

4. An X-ray apparatus as claimed in claim 2, wherein said supporting arrangement moves said C-arm through an angulation movement for acquiring said series of 2D projections.

5. An X-ray apparatus as claimed in claim 2 wherein said C-arm and said supporting arrangement form an isocentric apparatus.

6. An X-ray apparatus as claimed in claim 1 comprising a computer supplied with said series of 2D projections for calculating a volume dataset of the body of the examination subject, and for combining said image dataset with said volume dataset by a combination procedure selected from the group consisting of fusing and superimposing.

7. A method comprising the steps of:
disposing an examination subject in an x-ray imaging system comprising a carrier support with an x-ray source and a radiation detector mounted thereon at respective positions allowing the examination subject to be disposed between the x-ray source and the radiation detector;
moving said carrier support relative to the examination subject for acquiring a series of 2D projections of the examination subject with the x-ray source and the radiation detector; and
with an optical 3D sensor mounted to said carrier support, also moving said carrier support relative to said examination subject for acquiring an image dataset with said optical 3D sensor representing at least a portion of a surface of the examination subject.

8. A method as claimed in claim 7, comprising employing a C-arm as said carrier support.

9. A method as claimed in claim 8, wherein said C-arm has a circumference, and comprising moving said C-arm along said circumference during acquisition of said series of 2D projections.

10. A method as claimed in claim 8, comprising moving said C-arm through an angulation movement for acquiring said series of 2D projections.

11. A method as claimed in claim 8 wherein said C-arm and said supporting arrangement form an isocentric apparatus.

12. A method as claimed in claim 7 comprising supplying a computer with said series of 2D projections and, in said computer, calculating a volume dataset of the body of the examination subject, and for combining said image dataset with said volume dataset by a combination procedure selected from the group consisting of fusing and superimposing.
